(19) United States
(12) Patent Application Publication
(10) Pub. No.: US 2024/0166616 A1
(43) Pub. Date: May 23, 2024

(54) PROCESS FOR PREPARING ALKYL-4-OXOTETRAHYDROFURAN-2-CARBOXYLATE

(71) Applicant: Bayer Aktiengesellschaft, Leverkusen (DE)

(72) Inventors: Anton LISHCHYNSKYI, Leverkusen (DE); Carlos MORIANA HERRAIZ, Leverkusen (DE); Günter HÖMBERGER, Leverkusen (DE); Mark James FORD, Leverkusen (DE)

(21) Appl. No.: 18/548,530

(22) PCT Filed: Feb. 28, 2022
(86) PCT No.: PCT/EP2022/054924

§ 371 Date: Aug. 31, 2023

(30) Foreign Application Priority Data
Mar. 3, 2021 (EP) 21160525.8

(51) Int. Cl. C07D 307/32

(52) U.S. Cl. CPC C07D 307/32

(57) ABSTRACT

The present invention relates to a novel method for preparing alkyl 4-oxotetrahydrofuran-2-carboxylate (I).

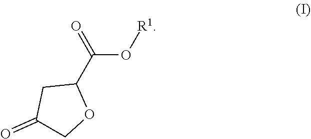

The present invention relates to a novel method for preparing alkyl 4-oxotetrahydrofuran-2-carboxylate (I).

Methyl 4-oxotetrahydrofuran-2-carboxylate of formula (I) is an important precursor of agrochemical (cf. WO2018/228985) active substances.

The synthesis of methyl 4-oxotetrahydrofuran-2-carboxylate of formula (I) is known, e.g. from Helv. Chim. Acta 1959, 1177 and WO 2016/205633. However, if starting from dimethyl (Z)-butenedioate, three reaction steps are necessary to prepare methyl 4-oxotetrahydrofuran-2-carboxylate of formula (I), which is accompanied by a loss of yield. Moreover, the reagents that are used in the prior art (for example sodium powder, NaH, TMSCHN₂, CH₂N₂) are unsuitable for an industrial-scale synthesis, since safe handling of these chemicals on a large scale is difficult or they are highly toxic.

In the light of the prior art described above, the object of the present invention is to develop, starting from compounds of general formula (II) and (III) in only two reaction steps, a method for preparing compounds of general formula (I) that is also suitable for production on a large scale.

The object described above is achieved by a method for preparing compounds of general formula (I)

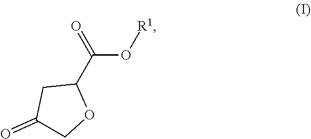

in which
R¹ is (C₁-C₄) alkyl,
characterized in that compounds of general formula (II)

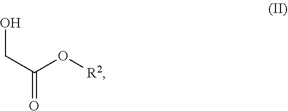

in which
R² is (C₁-C₄) alkyl,
with compounds of general formula (III)

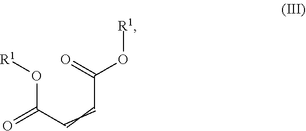

in which R¹ is as defined above,
through addition of MO^{t}Bu,
in which
M is an alkali metal ion,
cyclization products of general formula (IV)

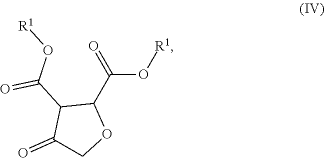

in which R¹ is as defined above,
which under non-hydrolytic conditions undergo dealkoxycarbonylation and react to form compounds of general formula (I).

Preferred definitions of the radicals for the compounds of general formulas (I), (II), (III), (IV) and MO^{t}Bu are as follows:
R¹ is ethyl or methyl,
R² is ethyl or methyl,
M is sodium or potassium.

Particularly preferred definitions of the radicals for the compounds of general formulas (I), (II), (III), (IV) and MO^{t}Bu are as follows:
R¹ is methyl,
R² is methyl,
M is sodium.

Elucidation of the Method

The reaction conditions for preparing compounds of general formula (I) are elucidated in detail hereinbelow.

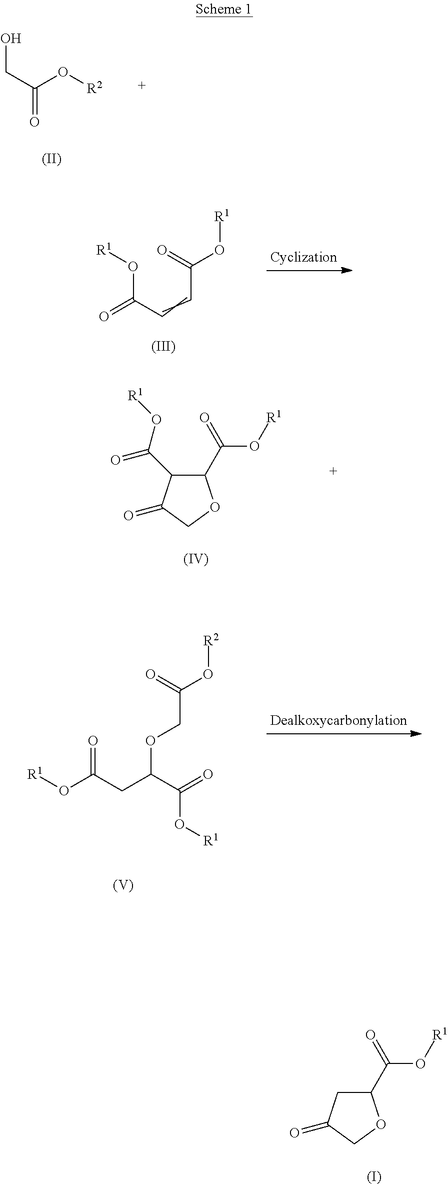

The compounds of general formula (II) react with compounds of general formula (III) in the presence of MO^{t}Bu to form cyclization products of general formula (IV), which under non-hydrolytic conditions undergo dealkoxycarbonylation and react to form compounds of general formula (I).

After the cyclization, in addition to the actual product, the compounds of general formula (IV), there may also be present in the reaction mixture residual reactant of general formula (III) and the intermediate of general formula (V).

The compounds of general formula (II) and (III) are commercially available. The compounds of general formula (III) can surprisingly be used in the form of the E isomer or the Z isomer. This is not known from the literature. Under the reaction conditions, an isomerization between the E isomer and the Z isomer takes place.

The compounds of general formula (I) have a stereocentre. The product is consequently present in the form of a racemate.

Cyclization:

The cyclization is known from the prior art, where it is carried out using NaH or sodium powder (Helv. Chim. Acta 1959, 1177; WO 2016/205633). These reagents are unsuitable for an industrial-scale synthesis, since their safe handling on a large scale is difficult.

The yield of the method according to the invention is higher (>30%) than that of the method using NaH or sodium powder described in the prior art (<30%). Moreover, the use of the tert-butoxide base means that the reaction can be employed on an industrial scale too.

Advantageous for achieving a high yield is the slow addition of MO^{t}Bu.

The addition of MO^{t}Bu takes place preferably over 0.5 to 8 hours, more preferably over 3 to 5 hours.

The molar ratio of MO^{t}Bu relative to compounds of general formula (II) is 0.8 to 3 equivalents, preferably 0.9 to 1.2 equivalents.

The molar ratio of compounds of general (II) to compounds of general formula (III) is 0.8 to 3 equivalents, preferably 0.9 to 1.2 equivalents.

The temperature may be varied within a wide range and depends for example on the solvent. It is for the reaction preferably 0° C. to 70° C., very particularly preferably 40° C. to 60° C.

The reaction is normally carried out in a solvent, preferably in THF, toluene or Me-THF. Preferably, this is an anhydrous (“dry” or absolute) solvent.

Dealkoxycarbonylation (Organic Reactions, Vol. 81):

Whereas with sulfuric acid in water (see Helv. Chim. Acta 1959, 1177; WO 2016/205633), ester cleavage/decarboxylation to 4-oxotetrahydrofurancarboxylic acid takes place, under non-hydrolytic, e.g. anhydrous, reaction conditions, the ester in compounds of general formula (I) remains present, which means there is no need for the additional step of renewed esterification, which in the prior art is carried out with CH₂N₂. There is consequently also no need for the numerous extractions necessary in the prior art of the 4-oxotetrahydrofurancarboxylic acid, which is difficult to isolate from water.

The yield can consequently be increased substantially (>95% versus 75% with sulfuric acid in the prior art). Toxic reagents such as diazomethane can be dispensed with.

The reagent (see Table 1) is used in excess, optionally in combination with a solvent. Preferably, the reagent is also used as the solvent.

The temperature for the reaction depends on the reagent/solvent.

Table 1 lists some of these reaction conditions by way of example, but without any limitation thereto.

TABLE 1
Dealkoxycarbonylation

| Conditions | | |
|---|---|---|
| (Reagent = solvent) | Temp./° C. | Time/hours |
| Acetic acid | 118 | 6 |
| Propionic acid | 140 | 18 |
| B(OH)₃ | 145-175 | 4 |

### EXAMPLES

The present invention is elucidated in more detail by the examples that follow, without restriction of the invention thereto.

Measurement Methods

The products were characterized by ¹H NMR.

### Example 1

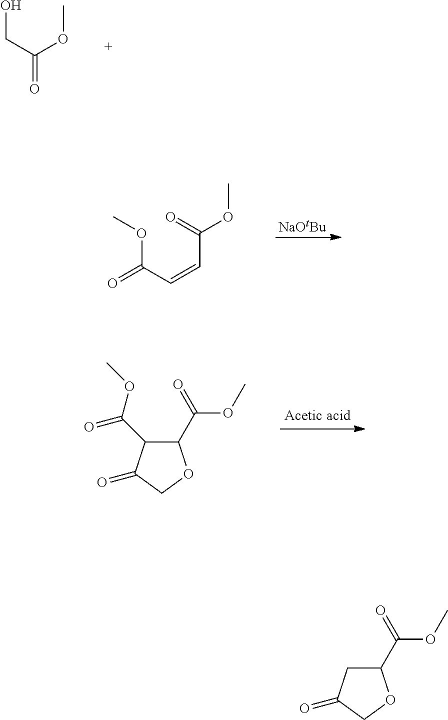

### Methyl 4-oxotetrahydrofuran-2,3-dicarboxylate

108 g of methyl glycolate (1.2 mol) and 172 g of dimethyl maleate (1.2 mol) are added together with 800 ml of THF to a vessel equipped with a heating/cooling jacket (2 l). The mixture is heated to 50° C. and then a solution of 120 g of NaO^{t}Bu (1.25 mol) in 800 ml of THF is added over a period of 3 hours. During the first minutes, the internal temperature rises to 53° C. and then remains at 50° C. After approx. 15% of the base has been added, the reaction mixture becomes turbid and a solid precipitates. At the end of addition of the base, the reaction mixture is stirred at 50° C. for 1 hour further and is then cooled to −1° C. To this is then added 215 g of acetic acid (3.6 mol) over a period of 20 minutes. The temperature of the reaction mixture rises to 6° C. The reaction mixture is then cooled to 1° C. and 47.1 g of HCl gas is introduced below the level of the liquid over a period of 45 minutes. The temperature of the reaction mixture rises to 8.5° C. The THF solvent is removed by distillation at a pressure of 50 mbar and a temperature of 50° C. What is left behind is an oil.

### Methyl 4-oxotetrahydrofuran-2-dicarboxylate

The oil from the previous reaction step is added to 200 ml of acetic acid and the solid produced is filtered off. The filtrate is heated to 118° C. for a period of 6 hours. The acetic acid is then distilled off at 50° C. and a pressure of 4 mbar. The product is purified by distillation. The product is an oil (87.4 g, 46%).

¹H NMR (600 MHz, DMSO-d6): δ (ppm)=4.97 (dd, J=8.8, 5.1 Hz, 1H), 4.02 (d, J=16.7 Hz, 1H), 3.99 (d, J=16.7 Hz, 1H), 3.69 (s, 3H), 2.88 (dd, J=18.2, 8.8 Hz, 1H), 2.60 (dd, J=18.2, 5.1 Hz, 1H).

TABLE 2
Comparison of the yield

| | Reaction conditions | Overall yield |
|---|---|---|
| Example No. 1 | NaO^{t}Bu/acetic acid | 46% |
| Comparative example: | Sodium powder, sulfuric | 23% |
| Helv. Chim. Acta 1959, 1177 | acid, water, CH₂N₂ | |

1. A method for preparing compounds of general formula (I) 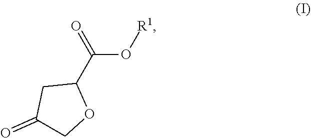
in which
R¹ is (C₁-C₄) alkyl,
characterized in that compounds of general formula (II) 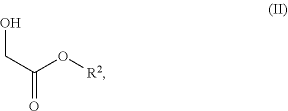
in which
R² is (C₁-C₄) alkyl,
are reacted with compounds of general formula (III) 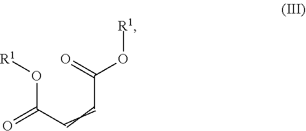
in which R¹ is as defined above,
in the presence of MO^{t}Bu,
in which
M is an alkali metal ion,
to form cyclization products of general formula (IV) 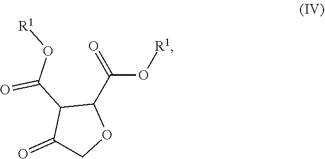
in which R¹ is as defined above,
which under non-hydrolytic conditions the cyclization products undergo dealkoxycarbonylation and react to form compounds of general formula (I).

2. The method according to claim 1, characterized in that:
R¹ is ethyl or methyl,
R² is ethyl or methyl,
M is sodium or potassium.

3. The method according to claim 2, characterized in that:
R¹ is methyl,
R² is methyl,
M is sodium.

4. The method according to claim 1, characterized in that the cyclization is carried out at 0° C. to 70° C.

5. The method according to claim 4, characterized in that the cyclization is carried out at 40° C. to 60° C.

6. The method according to claim 1, characterized in that a solvent used for the cyclization is THF, toluene or Me-THF.

7. The method according to claim 6, characterized in that the solvent is used for the cyclization is an anhydrous solvent.

8. The method according to claim 1, characterized in that MO^{t}Bu is added over 0.5 to 8 hours during the cyclization.

9. The method according to claim 1, characterized in that MO^{t}Bu is metered in during the cyclization.

10. The method according to claim 1, characterized in that a reagent/solvent in the dealkoxycarbonylation is AcOH.
